(19) United States
(12) Patent Application Publication
(10) Pub. No.: US 2005/0037047 A1
(43) Pub. Date: Feb. 17, 2005

(54) Medical devices comprising spray dried microparticles

(76) Inventor: Young-Ho Song, Framingham, MA (US)

Correspondence Address: MAYER, FORTKORT & WILLIAMS, PC, 251 NORTH AVENUE WEST, 2ND FLOOR, WESTFIELD, NJ, 07090, US

(21) Appl. No.: 10/638,564

(22) Filed: Aug. 11, 2003

(51) Int. Cl. A61F 13/00

(52) U.S. Cl. 424/423

(57) ABSTRACT

An implantable or insertable medical device which includes (a) a tacky polymeric region and (b) spray dried microparticles, which are adhered to the tacky polymeric region. The present invention is further directed to methods of forming such medical devices, and methods of releasing a therapeutic agent within a patient using such medical devices.

### FIELD OF THE INVENTION

The present invention relates to implantable or insertable medical devices for delivery of one or more therapeutic agents to a patient.

### BACKGROUND OF THE INVENTION

Numerous medical devices have been developed for the delivery of therapeutic agents to the body. In accordance with some delivery strategies, a therapeutic agent is provided within a polymeric release layer that is associated with an implantable or insertable medical device. Once the medical device is placed at a desired location within a patient, the therapeutic agent is released from the medical device. The release profile of the therapeutic agent is dependent upon a number of factors, including the specific condition being treated, the specific therapeutic agent selected, the specific site of administration, and so forth.

Therapeutic-agent-containing microparticles are also known in the pharmaceutical field. In some cases, the therapeutic agent is provided within a biodegradable or non-biodegradable matrix, in which case the microparticle is sometimes referred to as a “micromatrix,” while in other cases, the therapeutic agent is encapsulated within a biodegradable or non-biodegradable shell, in which case the microparticle is sometimes referred to as a “microcapsule.” Microparticles are useful for controlling drug release and therefore allow for the possibility of site-specific drug targeting. Microparticles can protect the therapeutic agents contained therein from premature bioinactivation, and incorporation of both hydrophilic and lipophilic drugs is possible. Microparticles are commonly between 0.1 and 1000 microns in largest dimension, and they are frequently spherical in shape and are therefore sometimes referred to as “microspheres,” although other shapes are possible.

### SUMMARY OF THE INVENTION

The use of drug-containing microparticles in connection with polymeric portions of implantable or insertable medical devices would be beneficial, for example, from the viewpoint of therapeutic agent protection and from the viewpoint of controlled and targeted therapeutic agent release. Unfortunately, polymeric portions of implantable or insertable medical devices are commonly formed in a fashion that is incompatible with microparticles.

For instance, solvent-based techniques are frequently used for forming polymeric layers on medical devices. Using these techniques, a polymeric layer can be formed on a medical device substrate by first dissolving one or more polymers of interest in a solvent system containing one or more organic solvents, and subsequently applying the resulting solution to a medical device substrate, e.g., by spraying or dipping. Unfortunately, many of the materials commonly used to form drug-containing microparticles, for example, poly(lactide-co-glycolide), are soluble in organic solvent systems. Consequently, if one were to add such drug-containing microparticles to a solution of this type in an attempt to incorporate the microparticles into a polymeric layer, the structure of the microparticles would be lost.

The present inventor, however, has overcome these and other difficulties by providing implantable or insertable medical devices that include (a) a tacky polymeric region and (b) spray dried microparticles, which are adhered to the tacky polymeric region.

The polymeric regions of the medical devices of the present invention can be made tacky in a number of ways. As one example, one or more tacky polymers can be provided within a polymeric region to render the polymeric region tacky. Examples of tacky polymers include polymers and copolymers that contain acrylate ester monomers, methacrylate ester monomers, olefin monomers and/or siloxane monomers.

The spray dried microparticles used in the medical devices of the present invention include one or more therapeutic agents and one or more carrier polymers. In many beneficial embodiments, the carrier polymer is a biodegradable polymer, for example, a poly(alpha-hydroxy acid) such as poly(D, L-lactide-co-glycolide). Examples of spray dried microparticles appropriate for the practice of the present invention include both microcapsules and micromatrices.

A wide variety of implantable or insertable medical devices can be provided in connection with the present invention, including catheters, guide wires, balloons, filters, stents, stent grafts, vascular grafts, vascular patches, and shunts. The implantable or insertable medical devices of the present invention can be adapted for implantation or insertion into a variety of bodily sites, including the coronary vasculature, peripheral vascular system, esophagus, trachea, colon, biliary tract, urinary tract, prostate and brain.

Other aspects of the present invention are directed to methods of releasing therapeutic agent within a patient by implanting or inserting a medical device like those above into the patient. For example, a vascular stent in accordance with the present invention can be inserted into the vasculature of a patient to prevent restenosis.

Still other aspects of the present invention are directed to methods of forming implantable or insertable medical devices. These methods include the steps of (a) providing an implantable or insertable medical device that includes a tacky polymeric region and (b) exposing the tacky polymeric region to spray dried microparticles, such that the microparticles become adhered to the tacky region of the medical device. For instance, in one particularly beneficial embodiment of the present invention, a medical device is made by a process that includes directing spray dried microparticles onto a tacky polymeric region of the medical device, without an intermediate microparticle collection step, for example, by placing the medical device directly into a spray drying apparatus.

One advantage of the present invention is that implantable or insertable medical devices can be provided, in which therapeutic agent is released from microparticles.

Another advantage of the present invention is that medical devices can be provided, in which drugs are protected from degradation and premature bio-inactivation to control drug release.

Another advantage of the present invention is that medical devices can be provided that exhibit controlled drug release in a sustained release pattern. Such release characteristics are usefull for treating a number of diseases and conditions, for example, restenosis.

Another advantage of the present invention is that medical devices can be provided, which allow for the possibility of site-specific drug targeting.

These and other embodiments and advantages of the present invention will become immediately apparent to those of ordinary skill in the art upon review of the Detailed Description and Claims to follow.

### BRIEF DESCRIPTION OF THE DRAWINGS

FIG. 1 is a schematic view illustrating an apparatus and process for providing drug-releasing stents, in accordance with an embodiment of the present invention.

### DETAILED DESCRIPTION OF THE INVENTION

According to one aspect of the present invention, an implantable or insertable medical device is provided, which contains: (a) a tacky polymeric region; and (b) spray dried microparticles, which contain at least one therapeutic agent and at least one carrier polymer, and which are adhered to the tacky polymeric region.

By “polymeric region” is meant a region, which contains at least one polymer. As the term is used herein, a substance or region is “tacky” if it is sufficiently sticky that spray dried microparticles will adhere to it upon contact. Therefore, a “tacky polymeric region” is a polymeric region to which spray dried microparticles adhere upon contact.

The tacky polymeric region can be present in the medical device in a number of configurations. For example, the polymeric region can correspond to the entirety of the medical device, or it can correspond to only a portion of the medical device. The portion of the medical device can be, for example, (a) one or more medical device layers (e.g., one or more coating layers), (b) one or more medical device components or portions thereof, and so forth.

In some embodiments, the medical devices of the present invention are further provided with a barrier region. A “barrier region” is a region that is disposed between a source of therapeutic agent (e.g., spray dried microparticles) and a site of intended release, which controls the rate at which the therapeutic agent is released. The barrier region is typically in the form of a layer, although other configurations are possible.

Preferred implantable or insertable medical devices for use in conjunction with the present invention include catheters (for example, renal or vascular catheters), guide wires, balloons, filters (e.g., vena cava filters), stents (including coronary vascular stents, cerebral, urethral, ureteral, biliary, tracheal, gastrointestinal and esophageal stents), stent grafts, cerebral aneurysm filler coils (including Guglilmi detachable coils and metal coils), vascular grafts, myocardial plugs, patches, pacemakers and pacemaker leads, heart valves, biopsy devices, or any coated substrate (which substrate can comprise, for example, glass, metal, polymer, ceramic and combinations thereof) that is implanted or inserted into the body, either for procedural use or as an implant, and from which therapeutic agent is released.

The medical devices for use in connection with the present invention include drug delivery medical devices that are used for either systemic treatment or for localized treatment of any mammalian tissue or organ. Non-limiting examples are tumors; organs including but not limited to the heart, coronary and peripheral vascular system (referred to overall as “the vasculature”, lungs, trachea, esophagus, brain, liver, kidney, bladder, urethra and ureters, eye, intestines, stomach, pancreas, ovary, and prostate; skeletal muscle; smooth muscle; breast; cartilage; and bone.

One particularly preferred medical device for use in connection with the present invention is a vascular stent that delivers therapeutic agent into the vasculature for the treatment of restenosis. As used herein, “treatment” refers to the prevention of a disease or condition, the reduction or elimination of symptoms associated with a disease or condition, or the substantial or complete elimination a disease or condition. Preferred subjects (also referred to as “patients”are vertebrate subjects, more preferably mammalian subjects and more preferably human subjects.

Although the medical device release characteristics that are ultimately of interest to the medical practitioner are the release characteristics subsequent to implantation or insertion (administration) into a subject, it is well known in the art to quantify release characteristics of a medical device using an experimental system that gives an indication of the actual release characteristics within the subject. For example, aqueous buffer systems are commonly used for testing release of therapeutic agents from vascular devices.

A wide variety of polymers are available for use in the polymeric regions of the medical devices of the present invention, including one or more of the following: polycarboxylic acid polymers and copolymers including polyacrylic acids; acetal polymers and copolymers; acrylate and methacrylate polymers and copolymers (e.g., n-butyl methacrylate); cellulosic polymers and copolymers, including cellulose acetates, cellulose nitrates, cellulose propionates, cellulose acetate butyrates, cellophanes, rayons, rayon triacetates, and cellulose ethers such as carboxymethyl celluloses and hydoxyalkyl celluloses; polyoxymethylene polymers and copolymers; polyimide polymers and copolymers such as polyether block imides, polyamidimides, polyesterimides, and polyetherimides; polysulfone polymers and copolymers including polyarylsulfones and polyethersulfones; polyamide polymers and copolymers including nylon 6,6, polycaprolactams and polyacrylamides; resins including alkyd resins, phenolic resins, urea resins, melamine resins, epoxy resins, allyl resins and epoxide resins; polycarbonates; polyacrylonitriles; polyvinylpyrrolidones (cross-linked and otherwise); polymers and copolymers of vinyl monomers including polyvinyl alcohols, polyvinyl halides such as polyvinyl chlorides, ethylene-vinylacetate copolymers (EVA), polyvinylidene chlorides, polyvinyl ethers such as polyvinyl methyl ethers, polystyrenes, styrene-maleic anhydride copolymers, styrene-butadiene copolymers, styrene-ethylene-butylene copolymers (e.g., a polystyrene-polyethylene/butylene-polystyrene (SEBS) copolymer, available as Kraton® G series polymers), acrylonitrile-styrene copolymers, acrylonitrile-butadiene-styrene copolymers, styrene-butadiene copolymers and styreneisobutylene copolymers (e.g., polyisobutylene-polystyrene block copolymers such as SIBS), polyvinyl ketones, polyvinylcarbazoles, and polyvinyl esters such as polyvinyl acetates; polybenzimidazoles; ionomers; polyalkyl oxide polymers and copolymers including polyethylene oxides (PEO); glycosaminoglycans; polyesters including polyethylene terephthalates and aliphatic polyesters such as polymers and copolymers of lactide (which includes lactic acid as well as d-,1- and meso lactide), epsilon-caprolactone, glycolide (including glycolic acid), hydroxybutyrate, hydroxyvalerate, para-dioxanone, trimethylene carbonate (and its alkyl derivatives), 1,4-dioxepan-2-one, 1,5-dioxepan-2-one, and 6,6-dimethyl-1,4-dioxan-2-one (a copolymer of polylactic acid and polycaprolactone is one specific example); polyether polymers and copolymers including polyarylethers such as polyphenylene ethers, polyether ketones, polyether ether ketones; polyphenylene sulfides; polyisocyanates; polyolefin polymers and copolymers, including polyalkylenes such as polypropylenes, polyethylenes (low and high density, low and high molecular weight), polybutylenes (such as polybut-1-ene and polyisobutylene), poly-4-methyl-pen-1-enes, ethylene-alpha-olefin copolymers, ethylene-methyl methacrylate copolymers and ethylene-vinyl acetate copolymers; fluorinated polymers and copolymers, including polytetrafluoroethylenes (PTFE), poly(tetrafluoroethylene-co-hexafluoropropene) (FEP), modified ethylene-tetrafluoroethylene copolymers (ETFE), and polyvinylidene fluorides (PVDF); silicone polymers and copolymers; polyurethanes; p-xylylene polymers; polyiminocarbonates; copoly(ether-esters)such as polyethylene oxide-polylactic acid copolymers; polyphosphazines; polyalkylene oxalates; polyoxaamides and polyoxaesters (including those containing amines and/or amido groups); polyorthoesters; biopolymers, such as polypeptides, proteins, polysaccharides and fatty acids (and esters thereof), including fibrin, fibrinogen, collagen, elastin, chitosan, gelatin, starch, glycosaminoglycans such as hyaluronic acid; as well as blends and copolymers of the above.

Such polymers may be provided in a variety of configurations, including cyclic, linear and branched configurations. Branched configurations include star-shaped configurations (e.g., configurations in which three or more chains emanate from a single branch point), comb configurations (e.g., graft polymers having a main chain and a plurality of branching side chains), and dendritic configurations (e.g., arborescent and hyperbranched polymers). As noted above, the polymers can be formed from a single monomer (i.e., they can be homopolymers), or they can be formed from multiple monomers (i.e., they can be copolymers) that can be distributed, for example, randomly, in an orderly fashion (e.g., in an alternating fashion), or in blocks.

In some embodiments of the present invention, a thin layer of tacky material is deposited on the polymeric region to render it tacky. In other embodiments, the polymeric region itself is tacky.

For example, in some embodiments, a polymeric region can be provided that is in an incomplete state of cure and thereby retains some degree of tackiness. In such embodiments, cure of the polymeric region is typically completed subsequent to microparticle adhesion.

In other embodiments, the polymeric region provided with one or more polymers that are inherently tacky, even when cured. Examples of inherently tacky polymers are known and include homopolymers and copolymers containing methacrylate, acrylate, silicone or olefin monomers, for example, homopolymers and copolymers containing: acrylate or methacrylate ester monomers, such as methyl methacrylate, butyl acrylate, butyl methacrylate, cyclohexyl methacrylate, isooctyl acrylate, isooctyl methacrylate, 2-ethylhexyl acrylate, 2-ethylhexyl methacrylate, and isoborynyl methacrylate; olefin monomers, such as isobutylene, butene, butadiene and isoprene; dialkyl siloxane monomers, such as dimethylsiloxane; and so forth. Several examples of tacky polymers are described, for example, in U.S. patent application Ser. No. 20010019721, U.S. patent application Ser. No. 20010051782, U.S. patent application Ser. No. 20020107330 and U.S. patent application Ser. No. 20020192273, the disclosures of which are hereby incorporated by reference.

Block copolymers containing (a) one or more poly(vinyl aromatic) blocks, for example, blocks of polystyrene or poly(a-metbyl styrene), and (b) a one or more polyolefin blocks, for example, blocks of polyisobutylene, polybutadiene, polyisoprene or polybutene, are one beneficial family of tacky polymers for the practice of the present invention. These polymers include diblock copolymers (e.g., polystyrene-polyolefin copolymers), triblock copolymer (e.g., polystyrene-polyolefin-polystyrene copolymers), star block copolymers, graft copolymers, dendrimers, and so forth. Several polymers within this family, including polystyrene-polyisobutylene-polystyrene triblock copolymers (SIBS copolymers), are described in U.S. patent application Ser. No. 20020107330 entitled “Drug delivery compositions and medical devices containing block copolymer.”

The tacky polymeric regions of the devices of the present invention (which, as previously noted, can correspond to device coatings, device components, entire devices, etc.) can be formed using a number of known techniques.

For example, where the polymer(s) of polymeric region have thermoplastic characteristics, a variety of standard thermoplastic processing techniques can be used to form the polymeric region, including compression molding, injection molding, blow molding, spinning, vacuum forming and calendaring, as well as extrusion into sheets, fibers, rods, tubes and other cross-sectional profiles of various lengths. As one specific example, an entire stent structure can be extruded using the above techniques. As another example, a coating can be provided by extruding a coating layer onto a pre-existing stent. As yet another example, a coating can be co-extruded along with an underlying stent structure.

In other embodiments, the polymeric region is formed using solvent-based techniques in which components of the polymeric region are first dissolved in a solvent system that contains one or more solvent species, and the resulting mixture is subsequently used to form a polymeric region. Preferred solvent-based techniques include, but are not limited to, solvent casting techniques, spin coating techniques, web coating techniques, solvent spraying techniques, dipping techniques, techniques involving coating via mechanical suspension such as air suspension, ink jet techniques, electrostatic techniques, and so forth.

Where appropriate, techniques such as those listed above can be repeated or combined to build up a polymeric region to a desired thickness. The thickness of the polymeric region can be varied in other ways as well. As a specific example, in solvent spraying, thickness can be increased by modification of coating process parameters, including increasing spray flow rate, slowing the movement between the substrate to be coated and the spray nozzle, providing repeated passes and so forth.

In other embodiments, a polymeric region is formed from a semi-cured material. In these embodiments, a region of uncured or semi-cured material can be provided using a variety of techniques (for example, casting techniques, spin coating techniques, web coating techniques, spraying techniques, dipping techniques, techniques involving coating via mechanical suspension such as air suspension, ink jet techniques, electrostatic techniques, and so forth), followed by a partial curing step, if desired.

Once a tacky polymeric region is established, microparticles are exposed to the same, resulting in adhesion of the microparticles to the tacky polymeric region. In cases where the microparticles are adhered to an uncured or partially cured layer, the polymeric region is typically subjected to additional curing after adhesion.

Microparticles for use in connection with the present invention are preferably prepared using spray drying techniques, because these techniques are fast, they are simple, and they are capable of providing microparticles with high drug loadings. These methods are also capable of providing high drug encapsulation efficiency as well as limited or minimal exposure of the drug to harsh solvents.

In some embodiments of the present invention, previously formed and collected spray dried particles are adhered to the tacky polymeric layer. In other embodiments, the spray dried particles are adhered to the tacky polymeric region immediately after formation and prior to collection, thereby eliminating a process step.

Microparticle spray drying is a process in which a liquid mixture of an evaporable liquid (which can comprise one or more liquid species), one or more drugs, and one or more carrier polymers is directed into a drying gas to achieve a dry particulate composition.

The liquid mixture may be a solution, an emulsion, a suspension, or the like. As a general rule of thumb, the more homogeneous is the liquid mixture, the more uniform is the distribution of the components in the resulting microparticles. In many embodiments, the liquid mixture is a solution, as this provides a high degree of homogeneity.

The evaporable liquid can be formed from a wide range of evaporable species including, for example, water, water miscible and immiscible organic species such as acetone, methanol, ethanol, propanol, isopropanol, dichloromethane, tetrahydrofuran, toluene, and dimethylsulfoxide, and mixtures the same.

The carrier polymer(s) can be selected, for example, from the above polymers, and can be the same as, or different from, the polymers used in the formation of the tacky polymeric region. In some embodiments, a biodegradable material is used for the formation of the spray dried particles, while a biostable material (for example, a methacrylate-, acrylate-, silicone- or olefin-containing homopolymer or copolymer such as those discussed above) is used to form the polymeric region of the device.

Examples of biodegradable materials for the formation of spray dried particles include poly(alpha-hydroxy acids), for example, polylactic acid, polyglycolic acid and copolymers and mixtures thereof such as poly(L-lactide) (PLLA), poly(D,L-lactide) (PLA); poly(glycolide) (PGA), poly(L-lactide-co-D,L-lactide) (PLLA/PLA), poly(L-lactide-co-glycolide) (PLLA/PGA), poly(D, L-lactide-co-glycolide) (PLA/PGA), poly(glycolide-co-trimethylene carbonate) (PGA/PTMC), poly(D,L-lactide-co-caprolactone) (PLA/PCL), poly(glycolide-co-caprolactone) (PGA/PCL); polyethylene oxide (PEO); polydioxanone (PDS); polypropylene fumarate; poly(ethyl glutamate-co-glutamic acid); poly(tert-butyloxy-carbonylmethyl glutamate); poly(carbonate-esters); polycaprolactone (PCL) and copolymers thereof such as polycaprolactone co-butylacrylate; polyhydroxybutyrate (PHBT) and copolymers of polyhydroxybutyrate; poly(phosphazene); poly(phosphate ester); polypeptides; polydepsipeptides, maleic anhydride copolymers; polyphosphazenes; polyiminocarbonates; poly (dimethyl-trimethylene carbonate-co-trimethylene carbonate); polycyanoacrylate, polysaccharides such as hyaluronic acid; and copolymers and mixtures of the above polymers, among others.

The liquid mixture is typically atomized to form fine droplets using various schemes including pressure atomization, rotary atomization and two-fluid atomization. In two common schemes, the liquid mixture is pumped through an orifice, such as a nozzle, or sprayed through a spinning perforated disc.

No particular restrictions are placed on the gas used to dry the atomized liquid mixture. Typical gases include air, or an inert gas such as nitrogen or argon. A variety of liquid-gas contacting schemes are known, including co-current flow, counter-current flow, and a mixture of co-current flow and counter-current flow. Once atomized, the liquid evaporates from the atomized droplets forming microparticles.

The temperature of the inlet of the gas used to dry the atomized mixture is preferably elevated, but not so elevated that it causes heat deactivation of the sprayed material. However, because the particles never reach the temperature of the drying gas, degradation is lower than might otherwise be expected. Other process parameters such as outlet temperature, feed rate of the liquid mixture, feed rate of the drying gas, disk/nozzle configurations, etc. can also be adjusted, as is known in the art.

Equipment for spray drying liquid mixtures is readily available from a number of commercial suppliers, such as Buchi, Niro, Yamato Chemical Co., Okawara Kakoki Co., and the like. More information on spray drying can be found, for example, in U.S. patent application Ser. No. 20020065399, U.S. Pat. No. 6,479,049, U.S. Pat. No. 6,309,623, U.S. Pat. No. 5,985,309, U.S. Pat. No. 5,648,096, and http :/www.incineratorsystem.com/products2.htm

The microparticles that are produced can range widely is size, but for purposes of the present invention, they are typically composed of particles, the majority of which have diameters in the range of 1 to 100 microns.

As previously noted, and in accordance with an embodiment of the present invention, spray dried microparticles are brought into contact with a tacky polymeric region, resulting in the adhesion of the spray dried microparticles to the polymeric region. Although previously formed and collected spray dried particles can be adhered to the tacky polymeric layer, in many beneficial embodiments of the invention, the spray dried microparticles are adhered to the tacky polymeric region immediately after formation and without being collected.

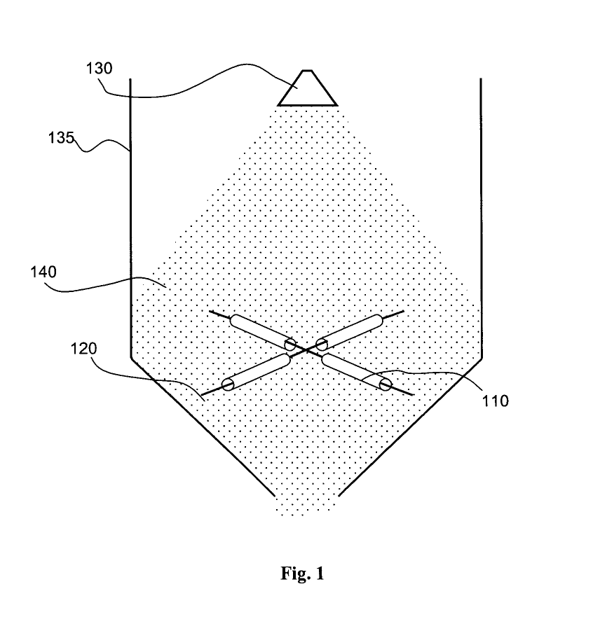

In this connection, a specific embodiment of the present invention will now be described with reference to FIG. 1. A number of stents 110 (one numbered), in this case, coronary stents, are provided with a tacky polymeric coating, for example, a polystyrene-polyisobutylene-polystyrene triblock copolymer (SIBS) coating, which can be produced and deposited in the manner discussed in U.S. patent application Ser. No. 20020107330 entitled “Drug delivery compositions and medical devices containing block copolymer.” The stents 110 with the tacky SIBS polymeric coating are mounted on a stent-holding apparatus 120 within a spraying chamber 135. As discussed above, a liquid mixture of drug (e.g., a drug targeting restenosis, such as paclitaxel) and a carrier polymer (e.g., a biodegradable carrier such as poly(D,L-lactide-co-glycolide) in an appropriate solvent system, is pumped through an atomizer 130. Upon contact with the drying gas in the spray drying apparatus (e.g., air), the solvent system is at least partially evaporated from the atomized droplets, forming microparticles 140. The newly formed microparticles 140, which may contain some residual solvent, thereafter contact the stents 110, where the microparticles 140 become adhered, due to the tacky nature of the surface of the stents 110. The stent-holding apparatus 120 is adapted to rotate the stents 110, to promote even coverage of the stents 110 with the microparticles 140.

A wide range of therapeutic agent loadings can be used in connection with the medical devices of the present invention, with the amount of loading being readily determined by those of ordinary skill in the art and ultimately depending, for example, upon the condition to be treated, the nature of the therapeutic agent itself, the means by which the therapeutic agent is administered to the intended subject, and so forth.

As previously noted, barrier layers can be formed over the microparticles, to further control the release of drugs from the same. In many embodiments, the barrier layer will comprise one or more polymers, which can be selected, for example, from the polymers described elsewhere in this application.

“Therapeutic agents”, “pharmaceutically active agents”, “pharmaceutically active materials”, “drugs” and other related terms may be used interchangeably herein and include genetic therapeutic agents, non-genetic therapeutic agents and cells. Therapeutic agents may be used singly or in combination. Therapeutic agents may be, for example, nonionic, or they may be anionic and/or cationic in nature.

Exemplary non-genetic therapeutic agents for use in connection with the present invention include: (a) anti-thrombotic agents such as heparin, heparin derivatives, urokinase, and PPack (dextrophenylalanine proline arginine chloromethylketone); (b) anti-inflammatory agents such as dexamethasone, prednisolone, corticosterone, budesonide, estrogen, sulfasalazine and mesalamine; (c) anti-neoplastic/antiproliferative/anti-miotic agents such as paclitaxel, 5-fluorouracil, cisplatin, vinblastine, vincristine, epothilones, endostatin, angiostatin, angiopeptin, monoclonal antibodies capable of blocking smooth muscle cell proliferation, and thymidine kinase inhibitors; (d) anesthetic agents such as lidocaine, bupivacaine and ropivacaine; (e) anti-coagulants such as D-Phe-Pro-Arg chloromethyl ketone, an RGD peptide-containing compound, heparin, hirudin, antithrombin compounds, platelet receptor antagonists, anti-thrombin antibodies, anti-platelet receptor antibodies, aspirin, prostaglandin inhibitors, platelet inhibitors and tick antiplatelet peptides; (f) vascular cell growth promoters such as growth factors, transcriptional activators, and translational promotors; (g) vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; (h) protein kinase and tyrosine kinase inhibitors (e.g., tyrphostins, genistein, quinoxalines); (i) prostacyclin analogs; (j) cholesterol-lowering agents; (k) angiopoietins; (l) antimicrobial agents such as triclosan, cephalosporins, aminoglycosides and nitrofurantoin; (m) cytotoxic agents, cytostatic agents and cell proliferation affectors; (n) vasodilating agents; and (o)agents that interfere with endogenous vasoactive mechanisms.

Exemplary genetic therapeutic agents for use in connection with the present invention include anti-sense DNA and RNA as well as DNA coding for: (a) anti-sense RNA, (b) tRNA or rRNA to replace defective or deficient endogenous molecules, (c) angiogenic factors including growth factors such as acidic and basic fibroblast growth factors, vascular endothelial growth factor, epidermal growth factor, transforming growth factor α and β, platelet-derived endothelial growth factor, platelet-derived growth factor, tumor necrosis factor α, hepatocyte growth factor and insulin-like growth factor, (d) cell cycle inhibitors including CD inhibitors, and (e) thymidine kinase (“TK”) and other agents useful for interfering with cell proliferation. Also of interest is DNA encoding for the family of bone morphogenic proteins (“BMP's”), including BMP-2, BMP-3, BMP-4, BMP-5, BMP-6 (Vgr-1), BMP-7 (OP-1), BMP-8, BMP-9, BMP-10, BMP-11, BMP-12, BMP-13, BMP-14, BMP-15, and BMP-16. Currently preferred BMP's are any of BMP-2, BMP-3, BMP-4, BMP-5, BMP-6 and BMP-7. These dimeric proteins can be provided as homodimers, heterodimers, or combinations thereof, alone or together with other molecules. Alternatively, or in addition, molecules capable of inducing an upstream or downstream effect of a BMP can be provided. Such molecules include any of the “hedgehog” proteins, or the DNA's encoding them.

Vectors for delivery of genetic therapeutic agents include (a) plasmids, (b) viral vectors such as adenovirus, adenoassociated virus and lentivirus, and (c) non-viral vectors such as lipids, liposomes and cationic lipids.

Cells for use in connection with the present invention include cells of human origin (autologous or allogeneic), including stem cells, or from an animal source (xenogeneic), which can be genetically engineered, if desired, to deliver proteins of interest.

Numerous therapeutic agents, not necessarily exclusive of those listed above, have been identified as candidates for vascular treatment regimens, for example, as agents targeting restenosis. Such agents are useful for the practice of the present invention and include one or more of the following: (a) Ca-channel blockers including benzothiazapines such as diltiazem and clentiazem, dihydropyridines such as nifedipine, amlodipine and nicardapine, and phenylalkylamines such as verapamil, (b) serotonin pathway modulators including: 5-HT antagonists such as ketanserin and naftidrofuryl, as well as 5-HT uptake inhibitors such as fluoxetine, (c) cyclic nucleotide pathway agents including phosphodiesterase inhibitors such as cilostazole and dipyridamole, adenylate/Guanylate cyclase stimulants such as forskolin, as well as adenosine analogs, (d) catecholamine modulators including a-antagonists such as prazosin and bunazosine, β-antagonists such as propranolol and α/β-antagonists such as labetalol and carvedilol, (e) endothelin receptor antagonists, (f) nitric oxide donors/releasing molecules including organic nitrates/nitrites such as nitroglycerin, isosorbide dinitrate and amyl nitrite, inorganic nitroso compounds such as sodium nitroprusside, sydnonimines such as molsidomine and linsidomine, nonoates such as diazenium diolates and NO adducts of alkanediamines, S-nitroso compounds including low molecular weight compounds (e.g., S-nitroso derivatives of captopril, glutathione and N-acetyl penicillamine) and high molecular weight compounds (e.g., S-nitroso derivatives of proteins, peptides, oligosaccharides, polysaccharides, synthetic polymers/oligomers and natural polymers/oligomers), as well as C-nitroso-compounds, O-nitroso-compounds, N-nitroso-compounds and L-arginine,(g) ACE inhibitors such as cilazapril, fosinopril and enalapril, (h) ATII-receptor antagonists such as saralasin and losartin, (i) platelet adhesion inhibitors such as albumin and polyethylene oxide, (j) platelet aggregation inhibitors including aspirin and thienopyridine (ticlopidine, clopidogrel) and GP IIb/IIIa inhibitors such as abciximab, epitifibatide and tirofiban, (k) coagulation pathway modulators including heparinoids such as heparin, low molecular weight heparin, dextran sulfate and β-cyclodextrin tetradecasulfate, thrombin inhibitors such as hirudin, hirulog, PPACK(D-phe-L-propyl-L-arg-chloromethylketone) and argatroban, FXa inhibitors such as antistatin and TAP (tick anticoagulant peptide), Vitamin K inhibitors such as warfarin, as well as activated protein C, (l) cyclooxygenase pathway inhibitors such as aspirin, ibuprofen, flurbiprofen, indomethacin and sulfinpyrazone, (m) natural and synthetic corticosteroids such as dexamethasone, prednisolone, methprednisolone and hydrocortisone, (n) lipoxygenase pathway inhibitors such as nordihydroguairetic acid and caffeic acid, (o) leukotriene receptor antagonists, (p) antagonists of E- and P-selectins, (q) inhibitors of VCAM-1 and ICAM-1 interactions, (r) prostaglandins and analogs thereof including prostaglandins such as PGE1 and PGI2 and prostacyclin analogs such as ciprostene, epoprostenol, carbacyclin, iloprost and beraprost, (s) macrophage activation preventers including bisphosphonates, (t) HMG-CoA reductase inhibitors such as lovastatin, pravastatin, fluvastatin, simvastatin and cerivastatin, (u) fish oils and omega-3-fatty acids, (v) free-radical scavengers/antioxidants such as probucol, vitamins C and E, ebselen, trans-retinoic acid and SOD mimics, (w) agents affecting various growth factors including FGF pathway agents such as bFGF antibodies and chimeric fusion proteins, PDGF receptor antagonists such as trapidil, IGF pathway agents including somatostatin analogs such as angiopeptin and ocreotide, TGF-β pathway agents such as polyanionic agents (heparin, fucoidin), decorin, and TGF-β antibodies, EGF pathway agents such as EGF antibodies, receptor antagonists and chimeric fusion proteins, TNF-α pathway agents such as thalidomide and analogs thereof, Thromboxane A2 (TXA2) pathway modulators such as sulotroban, vapiprost, dazoxiben and ridogrel, as well as protein tyrosine kinase inhibitors such as tyrphostin, genistein and quinoxaline derivatives, (x) MMP pathway inhibitors such as marimastat, ilomastat and metastat, (y) cell motility inhibitors such as cytochalasin B, (z) antiproliferative/antineoplastic agents including antimetabolites such as purine analogs (e.g., 6-mercaptopurine or cladribine, which is a chlorinated purine nucleoside analog), pyrimidine analogs (e.g., cytarabine and 5-fluorouracil) and methotrexate, nitrogen mustards, alkyl sulfonates, ethylenimines, antibiotics (e.g., daunorubicin, doxorubicin), nitrosoureas, cisplatin, agents affecting microtubule dynamics (e.g., vinblastine, vincristine, colchicine, paclitaxel and epothilone), caspase activators, proteasome inhibitors, angiogenesis inhibitors (e.g., endostatin, angiostatin and squalamine), rapamycin, cerivastatin, flavopiridol and suramin, (aa) matrix deposition/organization pathway inhibitors such as halofuginone or other quinazolinone derivatives and tranilast, (bb) endothelialization facilitators such as VEGF and RGD peptide, and (cc) blood rheology modulators such as pentoxifylline.

Numerous additional therapeutic agents useful for the practice of the present invention are also disclosed in U.S. Pat. No. 5,733,925 assigned to NeoRx Corporation, the entire disclosure of which is incorporated by reference.

Although various embodiments are specifically illustrated and described herein, it will be appreciated that modifications and variations of the present invention are covered by the above teachings and are within the purview of the appended claims without departing from the spirit and intended scope of the invention.

1. An implantable or insertable medical device comprising: (a) a tacky polymeric region; and (b) spray dried microparticles comprising a therapeutic agent and a carrier polymer; wherein said spray dried microparticles are adhered to said tacky polymeric region.

2. The implantable or insertable medical device of claim 1, wherein said tacky polymeric region comprises a tacky polymer.

3. The implantable or insertable medical device of claim 2, wherein said tacky polymeric region comprises two or more tacky polymers.

4. The implantable or insertable medical device of claim 2, wherein said tacky polymer is a polymer or copolymer comprising a monomer selected from acrylate ester monomers, methacrylate ester monomers, olefin monomers and siloxane monomers.

5. The implantable or insertable medical device of claim 2, wherein said tacky polymer is a polymer or copolymer comprising a monomer selected from methyl methacrylate, butyl acrylate, butyl methacrylate, cyclohexyl methacrylate, isooctyl acrylate, isooctyl methacrylate, 2-ethylhexyl acrylate, 2-ethylhexyl methacrylate, isoborynyl methacrylate, isobutylene, butene, butadiene, isoprene, and dimethylsiloxane.

6. The implantable or insertable medical device of claim 2, wherein said tacky polymer is a block copolymer comprising a poly(vinyl aromatic) block and a polyolefin block.

7. The implantable or insertable medical device of claim 6, wherein said poly(vinyl aromatic) block is selected from a polystyrene block and a poly(α-methyl styrene) block and wherein said polyolefin block is selected from a polyisobutylene block, a polybutadiene block, a polyisoprene block and a polybutene block.

8. The implantable or insertable medical device of claim 7, wherein said tacky polymer is a polystyrene-polyisobutylene-polystyrene triblock copolymer.

9. The implantable or insertable implantable or insertable medical device of claim 1, wherein said tacky polymeric region is in the form of a coating layer on said medical device.

10. The implantable or insertable medical device of claim 1, wherein said spray dried microparticles are microcapsules.

11. The implantable or insertable medical device of claim 1, wherein said spray dried microparticles are micromatrices.

12. The implantable or insertable medical device of claim 1, wherein said carrier polymer is a biodegradable polymer.

13. The implantable or insertable medical device of claim 12, wherein said biodegradable polymer is a poly(alpha-hydroxy acid).

14. The implantable or insertable medical device of claim 12, wherein said biodegradable polymer is a polymer or copolymer of lactic acid or glycolic acid.

15. The implantable or insertable medical device of claim 12, wherein said biodegradable polymer is selected from poly(L-lactide), poly(D,L-lactide), poly(L-lactide-co-D,L-lactide), poly(glycolide), poly(L-lactide-co-glycolide), and poly(D, L-lactide-co-glycolide).

16. The implantable or insertable medical device of claim 1, wherein said microparticles comprise two or more carrier polymers.

17. The implantable or insertable medical device of claim 1, further comprising a barrier coating over the microparticles.

18. The implantable or insertable medical device of claim 1, wherein said implantable or insertable medical device is selected from a catheter, a guide wire, a balloon, a filter, a stent, a stent graft, a vascular graft, a vascular patch, and a shunt.

19. The implantable or insertable medical device of claim 1, wherein said implantable or insertable medical device is adapted for implantation or insertion into the coronary vasculature, peripheral vascular system, esophagus, trachea, colon, biliary tract, urinary tract, prostate or brain.

20. The implantable or insertable medical device of claim 1, wherein said therapeutic agent is selected from one or more of the group consisting of an anti-thrombotic agent, an anti-proliferative agent, an anti-inflammatory agent, an anti-migratory agent, an agent affecting extracellular matrix production and organization, an anti-neoplastic agent, an anti-mitotic agent, an anesthetic agent, an anti-coagulant, a vascular cell growth promoter, a vascular cell growth inhibitor, a cholesterol-lowering agent, a vasodilating agent, and an agent that interferes with endogenous vasoactive mechanisms.

21. A method of forming the medical device of claim 1, comprising spray drying said microparticles onto said tacky polymeric region, without an intermediate microparticle collection step.

22. A method of releasing a therapeutic agent within a patient comprising (a) providing the implantable or insertable medical device of claim 1 and (b) implanting or inserting the implantable or insertable medical device into a patient.

23. The method of claim 22, wherein said medical device is inserted into the vasculature.

24. The method of claim 23, wherein said therapeutic agent is released in the treatment of restenosis.
